(19) United States
(12) Patent Application Publication
(10) Pub. No.: US 2005/0004815 A1
(43) Pub. Date: Jan. 6, 2005

(54) Appointment scheduling using time segmented solution propositions

(75) Inventor: Geert Machtelinck, Wondelgem (BE)

Correspondence Address: LEYDIG VOIT & MAYER, LTD, TWO PRUDENTIAL PLAZA, SUITE 4900, 180 NORTH STETSON AVENUE, CHICAGO, IL, 60601-6780, US

(73) Assignee: QUADRAT, Mortsel (BE)

(21) Appl. No.: 10/882,776

(22) Filed: Jul. 1, 2004

(30) Foreign Application Priority Data
Jul. 1, 2003 (EP) 03101962.3

(51) Int. Cl. G06F 17/60; G21K 1/12; H05G 1/60; A61B 6/00; G01N 23/00

(52) U.S. Cl. 705/2; 378/4; 705/9

(57) ABSTRACT

A system and a method are described for scheduling appointments for e.g. patients in medical institutions for performing a procedure such as an examination, according to operational constraints and availability preference criteria. A problem arises when a user of the system gets only one or more available time slots for the appointment, having the duration of the procedure, instead of all possible solutions for a complete time window. A solution is to provide time segments, having a duration longer than or equal to the time slot, and giving the patient the option to select a start time within one of these time segments. The method may be used in a computer or server system e.g. for scheduling examinations of patients in a radiology department or for documentation purposes of these examinations.

### FIELD OF THE INVENTION

The present invention relates to a system and method for planning and scheduling appointments in time, more specifically for automated patient appointment scheduling in medical institutions, taking into account operational constraints and availability preference criteria.

### BACKGROUND OF THE INVENTION

The product named QPlanner C/S and distributed by “Quadrat, naamloze vennootschap” and “AGFA-GEVAERT, naamloze vennootschap”, both incorporated in Belgium, is a client/server based medical appointment scheduling system. QPlanner C/S provides a user interface where the system proposes possible appointments for the first possible day where an examination or group of examinations can take place. All possible appointments for that day are listed. The user is able to choose one of the given solutions and book the appointment, or let the system search for possible appointments starting from the next day. The user has, at no time, any overview of all possible solutions for the given request. In the current system, the user is expected to interact with the program to scroll over the days until he identifies an appointment that seems appropriate.

Another product is “UltraGendaPro”, distributed by the company “UltraGenda”. In the user interface of UltraGendaPro, the first n solutions in time are given. In case a new set of n solutions is requested, a new search has to be launched to the server. If several new searches have to be made, this is time-consuming for both user as customer.

This set of n solutions is presented as discrete solutions, and therefore limits the user's choice for a solution. An additional problem is that the solutions are listed as for their first availability in time. Other constraints such as resource preference, day preference, location preference, etc. are not used in the proposed solution presentation.

Another problem arises when a patient gets only one or more available time slots, wherein each slot has the duration of the procedure, such as an examination by a CT scanner. Most often, these time slots are disjunctive, i.e. the intersection of the available time slots is empty.

### SUMMARY OF THE INVENTION

The above-mentioned problems are solved by a method having the specific features set out in claims 1 and 8 and by a system having the specific features set out in claim 7. Specific features for preferred embodiments of the invention are set out in the dependent claims.

### BRIEF DESCRIPTION OF THE DRAWING

FIG. 1 shows an overview of solutions, presented as a list of segments of time.

FIG. 2 shows time blocks for scheduling an appointment.

FIG. 3 shows another configuration of time blocks for scheduling an appointment.

FIG. 4 shows an intersection of available time segments.

### DETAILED DESCRIPTION OF THE INVENTION

While the present invention will hereinafter be described in connection with preferred embodiments thereof, it will be understood that it is not intended to limit the invention to those embodiments.

Scheduling an appointment may relate to:
1. person or persons involved in the appointment; or,
2. devices required for the appointment; or,
3. the time for the appointment; or,
4. the location for the appointment; or,
5. any combination of person, device, time and location.

The “time” of the appointment is usually a time slot, i.e. a period in time that may be defined by a starting time and an-ending time. The “time” of the appointment, may also relate to a plurality of such time slots.

According to the current invention, a time period or a time window may be defined or specified by the user of the system, e.g. by entering a start time and an end time of the time window via the keyboard of a computer system. This computer system may be a client system, a server system or a stand-alone computer system, which may be connected via a network to other computer systems. Once the time window is entered in the system, it is stored in a storing device for later retrieval or calculations. The user may for example specify that he wants a clinical procedure or examination during the months of August or October, because the patient is unavailable during September. In such case, the time window would be the union of the two available personal time segments 1 August up to and including 31 August and 1 October up to and including 31 October. As such, a time window may be fragmented in time segments. This time window may also exclude each period comprising a Saturday and Sunday. The time window may also specify that the patient is unavailable every morning before e.g. 1 p.m. The unavailability of the patient may alternatively also be coded in the “constraints”.

A time segment is a period of time within the selected or specified time window. A time segment is preferably a period of time for which constraints and preferences may be evaluated simultaneously. If for example a piece of equipment, such as a CT scanner, is available between 9 and 12 a.m. and a physician, who has to be present during the examination, is available between 8 and 11 a.m., it may be advantageous to define a time segment from 9 to 11 a.m., which is the time period during which the CT scanner and the physician are simultaneously available.

A time window may be typically some days to some months, whereas a time segment may be typically some hours, or a full working day. Usually, at least one or even most of the time segments have a duration that is longer than the time required for the examination or medical procedure.

In a preferred embodiment, a time segment is available, i.e. referred to as an “available time segment” if it obeys all constraints. In such situation, a time segment is preferably not offered to the user of the system, if one of the constraints has not been fulfilled or is not met. A first type of constraint may relate to the management of a resource. This means that the resource must be available during the duration of the medical procedure or examination. Another constraint may be the duration of a medical is procedure or examination. It is clear that, if a procedure takes one hour, it can not be planned in a period where a resource, such as a CT scanner, is available only for fifteen minutes. Another constraint may be the availability of a resource. If a specific medical device needs preventive maintenance e.g. every Tuesday, an examination, making use of this device, can not be planned during that day. Time segments for that device, referred to as “available resource time segments” will thus be absent for those days, or replaced by “unavailable” time segments, optionally indicating the reason for the unavailability. Also, if a device has already been allocated previously for a certain time slot, the available resource time segments relating to this device preferably do not overlap with that time slot. A specific time constraint may be that a certain physician, also generally related to as a resource, may be available only on Wednesday morning. The available time segments, requiring this resource, will thus mention Wednesday mornings only. The time segments for a resource may thus comprise “available resource time segments” and “unavailable” resource time segments. The unavailable time segments may e.g. specify that:
1. the time segment is outside normal working hours, e.g. during the night
2. the time segment is during a holiday period of a person who is required as a resource
3. the time segment has been already allocated for another appointment. The creation of these time segments may be done automatically by booking appointments for other patients.

The system may also be implemented such that each resource has an associated time window, defining when it is available. The time window for the above example will be “every Wednesday morning”. As soon as a time slot for an examination has been taken by a user, e.g. on one Wednesday morning from 9 to 10 a.m., then the time window for this resource is changed, to reflect the non-availability on that day from 9 to 10 a.m.

In a preferred embodiment, the time segments for the appointment are selected such that for each time segment all applicable constraints are fulfilled. In most cases a medical procedure can be scheduled if a room, a person and a device is available. A room may be selected from a group of rooms, a person may be selected from a group of persons, having substantially the same qualifications and a device may be selected from a group of devices, having substantially the same quality and capabilities. A group of substantially equivalent resources is called a resource group. A resource group may contain one or a plurality of similar or equivalent resources. For example, three CT scanners, having substantially the same capabilities, may form a “CT resource group”. A team of four physicians, having substantially the same qualifications, e.g. being a radiologist, may define a “radiologist resource group”.

If a patient needs a specific medical examination, this type of examination may require one member of a CT resource group, comprising 3 CTs i.e. CT1, CT2 and CT3 and one member of a radiologist resource group, comprising 4 radiologists, i.e. R1, R2, R3 and R4. In order to find all available time segments, during which the appointment can be scheduled, the system preferably proceeds as follows. The system first computes all possible combinations of CTs and radiologists, which are twelve in this situation, i.e. CT1-R1 (CT1=CT number 1; R1=radiologist number 1), CT1-R2, CT1-R3, CT1-R4, CT2-R1, CT2-R2, CT2-R3, CT2-R4, CT3-R1, CT3-R2, CT3-R3, CT3-R4. At least for one or some, or preferably for each individual combination, e.g. CT2-R3, the system will then generate all available time segments within the time window specified by the user. An available time segment in this example is a time segment when both a CT device and a radiologist are available, within the time window of the patient.

For this combination CT2-R3, the procedure may go as follows. The user enters in a computer system, such as a client system coupled to a server, the time window within which the available time segments must be located. This time window may specify two weeks, such as 2004.03.01 08:00 to 2004.03.14 18:00. The time window thus starts at Mar. 1, 2004 8 a.m. and ends on Mar. 14, 2004 6 p.m. The user enters in the computer system the medical examination type (“exam”), which reveals that this exam needs a first resource CTj from a first resource group CT1, CT2, CT3 and that this exam also needs a second resource Rk from a second resource group R1, R2, R3, R4. The system then makes all possible combinations CTj-Rk, as set out before, and selects them one by one. It is now possible that a specific combination, e.g. CT2-R2 is not allowed, for specific reasons. Exclusion of CT2-R2 means that radiologist R2 can not perform an examination in conjunction with CT system CT2. Combinations that are not allowed, are discarded from computing available time segments. The system will now, preferably for each allowed combination CTj-Rk compute the available time segments within the time window. For the combination CT2-R3, the available time segments are computed as follows. In the absence of the need for CT2 or R3, the initial available time segment for the exam is the segment:
2004.03.01 08:00-2004.03.14 18:00

This single contiguous time segment will now be adjusted, usually split in a series of contiguous time segments based on the availability of CT2 and R3.

Therefore, the availability of CT2 and R3 is consulted. The availability of CT2, globally referred to as “available resource time segments” may be stored in a second computer system, coupled to the first computer system of the user. The first and second computer system may be one single computer system, a client or a server system. The second computer system may be coupled to CT2. Preferably, the availability data for CT2 of this second computer system are accessible by all computer systems that need to allocate a time segment from CT2. Based upon the availability data for CT2, the initial single contiguous available time segment is split in contiguous time segments. Suppose that CT2 is available only on working days 1-5 and 8-12 March 2004 from 9 a.m. to 5 p.m. The availability data of CT2 also specify that a reservation on CT2 has been taken on March 2, from 10 a.m. to 11 a.m. Moreover, the data about the medical examination type specify that this exam requires the use of CT2 for minutes. If CT2 is available from 9 until 10 a.m., the first moment when the exam can start is 9 a.m. and the last moment when it can start is 9:45 a.m., since the exam with a duration of minutes will end at 10 a.m. As such, the intermediate available time segments are now:
2004.03.01 09:00-2004.03.01 16:45
2004.03.02 09:00-2004.03.02 09:45
2004.03.02 11:00-2004.03.02 16:45
2004.03.03 09:00-2004.03.03 16:45
2004.03.04 09:00-2004.03.04 16:45
2004.03.05 09:00-2004.03.05 16:45
2004.03.08 09:00-2004.03.08 16:45
2004.03.09 09:00-2004.03.09 16:45
2004.03.10 09:00-2004.03.10 16:45
2004.03.11 09:00-2004.03.11 16:45
2004.03.12 09:00-2004.03.12 16:45

From the above it is clear that from the end of each “available resource time segment” the duration of the medical examination is subtracted. This may be done consistently, such that the “available time segments” always reflect the moments at which an examination can start. This subtraction operation may also be done at the end of the calculations, i.e. each time when the solutions for one resource from a resource group or for one combination of resources from a plurality of resource groups is computed.

It is thus clear that the initial available time segment is fragmented in intermediate smaller available time segments, which take into account the time constraints of CT2, i.e. the “available resource time segments” for CT2.

In a further step, the availability of radiologist 3, i.e. R3 is consulted for imposing the time constraints of R3 on the intermediate available time segments. Suppose that R3 is only available during the first three days of the first week, i.e. 1-3 March 2004, and this from 8 a.m. to 11:45 a.m. Suppose also that radiologist R3 has been allocated for another exam already on March 2 from 9 to 10:15 a.m. The availability data for R3 are thus as follows:
2004.03.01 08:00-2004.03.01 11:45
2004.03.02 08:00-2004.03.02 09:00
2004.03.02 10:15-2004.03.02 11:45
2004.03.03 08:00-2004.03.03 11:45

These availability data for R3 may be stored in a third computer system, coupled to the first computer system, and these data are preferably accessible for all planning systems that need to plan resource R3.

The availability data for R3 are now combined with the intermediate available time segments, taking into account the duration 15 minutes for the medical examination. The latter means that the end time of each availability segment of R3 must be decreased with 15 minutes, for reflecting the acceptable start times of the examination, as to the availability of R3. Again, if the subtraction by the duration of the examination has not been done for the intermediate available time segments, then this is also not necessary at this stage for R3. In the list below, the intermediate available time segments are preceded by IM, whereas the R3 available start time segments are preceded by R3. The two types of time segments are merged below, to show how the system proceeds to compute the final available time segments. The intermediate time segments beyond 4 March are not shown, because no final available time segments can result. Underneath each pair of (partly) overlapping time segments for R3 and IM, the resulting available (“AV”) time segment is given:
R3 2004.03.01 08:00-2004.03.01 11:30
IM 2004.03.01 09:00-2004.03.01 16:45
AV 2004.03.01 09:00-2004.03.01 11:30
R3 2004.03.02 08:00-2004.03.02 08:45
IM 2004.03.02 09:00-2004.03.02 09:45
AV No overlap
R3 2004.03.02 10:15-2004.03.02 11:30
IM 2004.03.02 11:00-2004.03.02 16:45
AV 2004.03.02 11:00-2004.03.02 11:30
R3 2004.03.03 08:00-2004.03.03 11:30
IM 2004.03.03 09:00-2004.03.03 16:45
AV 2004.03.03 09:00-2004.03.03 11:30
IM 2004.03.04 09:00-2004.03.04 16:45
AV No overlapping data with R3

As can be extracted from the above data, the final available time segments for scheduling the start of the medical procedure are:
AV 2004.03.01 09:00-2004.03.01 11:30
AV 2004.03.02 11:00-2004.03.02 11:30
AV 2004.03.03 09:00-2004.03.03 11:30

In the above example, the overlap between R3 and IM was just pair-wise. In a more complex situation, a time segment of R3 can have an overlap with two or more subsequent time segments of IM or vice versa. To avoid missing final available time segments from the combination of intermediate available time segments (“IMj”) and available time segments for the resource (“REk”), the following procedure can be followed.

IMj for j=1, i.e. IM1 is compared with REk for k=1 . . . K, wherein K is the total number of time segments REk. The time segments REk have been reduced by the duration of the examination procedure.

Each time segment IMj has a start time IMj.S and an end time IMj.E. Each time segment REk has a start time REk.S and an end time REk.E. It is clear that IMj.S≦IMj.E and REk.S≦REk.E. The following situations can now occur:

| | | | |
|---|---|---|---|
| 1. | | IMj.E < REk.S → | No solution |
| 2. | IMj.S < REk.S and | REk.S ≦ IMj.E ≦ REk.E → | [REk.S , IMj.E] |
| 3. | IMj.S ≦ REk.S and | REk.E < IMj.E → | [REk.S , REk.E] |
| 4. | REk.S ≦ IMj.S ≦ REk.E and REk.S ≦ | IMj.E ≦ REk.E → | [IMj.S , IMj.E] |
| 5. | REk.S ≦ IMj.S ≦ REk.E and | REk.E < IMj.E → | [IMj.S , REk.E] |
| 6. | | REk.E < IMj.S → | No solution |

In situation 1, IMj is completely anterior to REk, such that there is no overlap.

In situation 2, the start of IMj is anterior to REk, however the end of IMj is within REk. Therefore, the solution or overlap starts at the start of REk and stops at the end of IMj.

In situation 3, REk is completely inside IMj. such that the solution is REk.

In situation 4, IMj is completely inside REk, such that the solution is IMj.

In situation 5, the start of IMj is within REk but the end of IMj is outside REk. Therefore, the solution or overlap starts at the start of IMj and stops at the end of REk.

In situation 6, IMj is completely posterior REk, such that there is no solution.

In one embodiment, each intermediate time segment IMj (j=1 . . . J) is compared to each available resource time segment REk (k=1 . . . K), preferably starting by comparing IM1 to RE1, RE2, . . . REK sequentially, and then proceeding by comparing IM2 to RE1, RE2, . . . REK until IMJ is compared to RE1, RE2, . . . REK. Each time when a solution is generated according to one of the above situations 2-5, this solution is stored in a new array of available time segments AVn. Such array may be a linked list of available time segments. This new array AVn will generally be more fragmented than the array of intermediate time segments IMj if the available resource time segments are highly fragmented. Moreover, the union of the available time segments AVn will be a subset of the union of the intermediate time segments IMj. If the intermediate time segments IMj and the resource time segments REk are sorted chronologically, the available time segments AVn will also be generated in a chronological order by this procedure. If IMj and REk are sorted chronologically, other optimisations of the comparison procedure are achievable, such that for each iteration IMj, not all resource time segments REk must be compared to the intermediate segment IMj. E.g. if IMj is found to be posterior to REk, then IMj+1 is more posterior to REk, and the comparison between IMj+1, IMJ+2, etc. and REk must not be made any more. A further comparison can start from REk+1. If IMj is found to be anterior to REk, then IMj is also anterior to REk+1, REk+2, etc., such that the iteration for comparing IMj to REk may be stopped at that time.

If the availability of time segments depends on a further resource, the same procedure as described herein above can be executed, where the obtained time segments AVn are treated as intermediate time segments IMj and the available time segments of this further resource are treated as resource time segments REk.

As soon as all time constraints of all required resources have been taken into account, the resulting array AVn gives the available time segments for that specific medical examination for the specific combination of resources, e.g. CT2 and R3, from resource groups.

The above procedure is now repeated for any other viable combination of a CT apparatus from the CT resource group and a radiologist R from the radiologist resource group.

Finally, the list of available time segments is formed by the union of AVn for any viable combination of resources in resource groups. If there is only one resource group, then the above computations have to be done only for each resource in the single resource group. In case there is more than one resource group, from each of which one resource must be selected, the computation is done for each viable combination of resources.

The final list of available time segments is the union of the available time segments found for the individual resources of one resource group or for the individual combinations of resources from two or more resource groups. These final available time segments are shown to the user, preferably on a display of the computer system at which the user performs the scheduling, and he can select, by using the keyboard or a pointer device such as a mouse or trackball, whatever moment in time, i.e. start time for the medical examination, within one of these available time segments. The system will then look up in the AVn lists, for which resource of the resource group or combination of resources from various resource groups the selected start time for the medical examination is appropriate. If the selected start time is compatible with various AVn lists or combinations, the user can be given the option to select one of these combinations. For conciseness of the description, “(resource) combination” is used both for one resource from one resource group or for a viable combination of two or more resources from two or more resource groups. Alternatively, the system can automatically select the most appropriate viable resource combination from the group. This selection may be done on an arbitrary basis, e.g. by just selecting the first viable resource combination. Alternatively, this selection may be done by providing a uniform load or occupation on all viable resources. It is also possible to define a maximum occupation for each resource, and to select that viable resource, which did not reach the maximum occupation yet. Once these selections are made, the user can “book” the appointment. Booking of the appointment will have the effect that at least one available resource time segment for the selected resource or resources is changed, with the effect that a time segment starting at the selected start time and having a duration dictated by the duration of the medical examination is declared as being unavailable. This time segment, unavailable for the resource, may get an attribute indicating a booking reference number, which points to all the particulars of the booking operation, or the appointment currently booked.

Some of the clinical examinations require not only the time of the examination itself, but also a “pre-op” and a “post-op” period. If a patient has to undergo an examination of 20 minutes using a main resource, it is possible that he needs 15 minutes of preparation before the examination, which is referred to as “pre-op”, and that he needs 10 minutes after the examination, which is referred to as “post-op”. It is clear that the main resource needs to be allocated for 20 minutes only. The patient however needs to be available 15 minutes before the examination will start and 10 minutes after the examination has been finished. These “pre-op” and “post-op” time segments can be used to define the initial time segments from the time window for the appointment. E.g. if the time window for the appointment is 2004.03.01 08:00 to 2004.03.14 18:00, as in the example above, the available time segment for the patient may be initialised as 2004.03.01 08:15 to 2004.03.14 17:50, to account for the pre-op and post-op respectively. If the pre-op or the post-op require specific other resources, such as a dressing room, also the availability of these resources may be considered when scheduling the appointment.

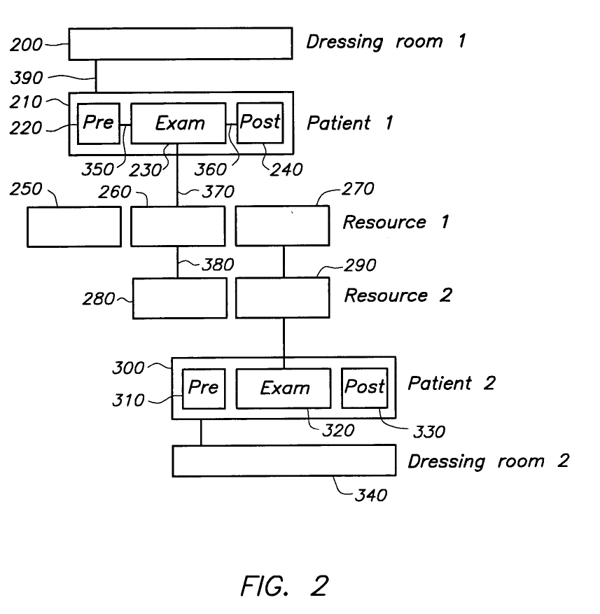

A more complex situation is shown in FIG. 2. The rectangular areas 200-340 represent time blocks for resources or patients. Area 210 represents the time that a patient must be available, not only for the examination 230 but also for the pre-op 220 and post-op 240. In the current example pre-op 220 and post-op 240 relate to undressing and dressing of the patient, required for the examination 230. Therefore, a dressing room 1, referred to by 200, is required from the start of-the pre-op until the end of the post-op. The duration of the pre-op 220 and the post-op 240 may be defined by the type of examination. The duration 230 of the examination is primarily defined by the type of examination, but may also depend on the resource or resources selected for this examination, i.e. an examination performed on a first device may take a longer time than the same type of examination, performed on a second equivalent device. The time blocks 250, 260 and 270 refer to allocations of a first resource “Resource 1” for performing an examination. The time blocks 280 and 290 refer to allocations of a second resource “Resource 2” for performing an examination. Time block 300 represents the scheduling of a second patient, requiring a pre-op 310, an examination 320 and a post-op 330. Time block 340 refers to the allocation of a second dressing room.

As set out below, the initialisation of available time segments and processing according to the above-mentioned time blocks leads to the correct scheduling of an appointment.

The initialisation of the available time segments is preferably done as follows. For each unit, i.e. resource, or patient, or dressing room, available time segments, corresponding to the time blocks in FIG. 2, are initialised by the “available personal time segments” or “available resource time segments”. Preferably, for each time block shown in FIG. 2, a linked list of time segments is created. Each element of the linked list may contain the start time when the unit is available and the end time when the unit is available. This linked list may now expand, e.g. by time segments fragmented by other units, or shrimp by deleting a time segment being incompatible with all time segments of another unit. During operation of the algorithm for searching a solution, individual elements of the linked list may get an increment of the start time or a decrement of the end time, such that the time segment gets a smaller duration. It is now necessary that time segments of interacting units correspond to each other. These interactions are defined by one, two or three types of links. These links define the structure of the full examination, i.e. all relations that may exist between the composing units involved in the examination. The structure defines which units are involved and their interrelations. Apart from the structure, the availability of the units, i.e. the available time segments, must be known, and the duration of the units, sketched by the time blocks in FIG. 2. In FIG. 2 the following links between time blocks are recognised:
1. Sequence links 350 and 360. Link 350 indicates that the exam 230 must immediately follow the pre-op 220. Link 360 indicates that the post-op 240 must immediately follow the exam 230.
2. Composition links. The time block 210 for patient 1 is composed of three time blocks, i.e. pre-op 220, exam 230 and post-op 240.
3. Relational links 370 and 380 for simultaneous actions. The exam 230 is linked via relational link 370 to the time block 260 of Resource 1. The time block 260 of resource 1 is linked via relational link 380 to the time block 280 of Resource 2. Alternatively, the exam 230 may be linked to time block 280, because the exam 230 needs resource 2 280.

The following conditions, if applicable, must now be fulfilled for having a solution:
1. The time segments for a unit must fulfil the availability of the unit. This is realised by the initialisation mentioned above.
2. The time segments of components (e.g. pre-op 220, exam 230, post-op 240) must be compatible with the time segments of the entity (e.g. “patient 1” 210) comprising these components.
3. A subsequent action (e.g. exam 230) must not start earlier than the earliest start of the previous action (e.g. pre-op 220) plus the duration of that previous action.
4. A previous action (e.g. exam 230) must not end later than the latest end of a following action (e.g. post-op 240) minus the duration of that following action.
5. Time segments for units with simultaneous actions must have equal start times and equal end times. This may be achieved by making the intersection of time segments of units having a relational link.

Each time segment indicates when the corresponding action may be executed. If time blocks are linked to each other via a relational link, then, graphically, the position and size of their corresponding time segments must match exactly, i.e. the start time and the end time of the time segments must match. As shown in FIG. 4, this means that if time block 81 is relationally linked to time block 82, then the start time of time segments 81 must be made equal to the start time of time segments 82 and the end time of time segments 81 must be made equal to the end time of time segments 82. FIG. 4 illustrates the situation where time segments 81 and 82 do not fulfil this condition. Time segments 81 may relate to the availability of the patient for performing the exam 230, whereas times segments 82 may relate to the availability of Resource 1. As can be seen in FIG. 4, the start points and end points of 81 and 82 do not coincide. Therefore, a new set of time segments 83 may be created, which is in fact the intersection of the time segments 81 and 82. This means that for the time segments 83, both 81 and 82 are available. After this intersection operation, the data representing time segments 81 for the patient and time segments 82 for Resource 1 may be overwritten by the time segments 83. For every link 370, 380 that time block 230 of Patient 1 has with time block 260 of Resource 1 and time block 280 of Resource 2 respectively, this operation is repeated, until all relational links for “Patient 1” have corresponding time segments.

As to the sequential links 350, 360 shown in FIG. 2, it is important to check that the start of any pre-op time segment is not earlier than the earliest start of a corresponding exam time segment minus the duration of pre-op. The end of pre-op time segment must not be later than the end of exam time segment minus the duration of exam 230. If one of these conditions is not met, because at least one of the time segments for exam 230,would have been changed in a previous operation, then the time segments for pre-op 220 must be re-adjusted.

Analogously, the start of time segments for post-op 240 must not be earlier than the start of the corresponding time segments for exam 230 plus the duration of the exam and the end of time segments for post-op 240 must not be later than the end of corresponding time segments of exam time block 230 plus the duration of the post-op 240. If one of these conditions is not met, because at least one of the time segments for exam 230 would have been changed in a previous operation, then the time segments for post-op 240 must be re-adjusted.

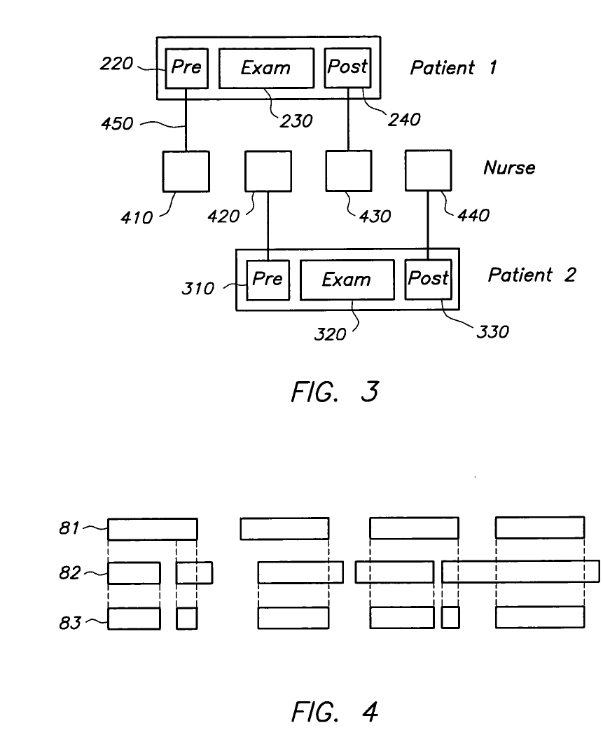

Once the time segments for pre-op 220 have been amended, this may influence other time segments, because of a corresponding relational link to other time blocks. This situation is shown in FIG. 3. It is possible that a pre-op 220 and post-op 240 of a patient needs the assistance of a nurse, shown by time blocks 410 and 430 respectively. Since there is a relational link 450 between pre-op 220 and nurse 410, the corresponding time segments must be made compatible, by taking the intersection of the available time segments corresponding to pre-op 220 and nurse 410, as discussed in conjunction with FIG. 4.

As to the composition links, which are shown in FIG. 2 as existing between Patient 1 (210) on one side and pre-op 220, exam 230 and post-op 240 on the other side, it is necessary that time segments having higher hierarchy, here Patient 1, start at the start of time segments of the first element of the lower hierarchy, here pre-op 220. The end of the time segments of the higher hierarchy must coincide with the end of the time segments of the last element of the lower hierarchy, here post-op 240. As such, if the time segments corresponding to time blocks 220 for pre-op have been amended, this may have an effect on the start time of the time segments corresponding to the time blocks 210 for Patient 1. Similarly, if the time segments corresponding to time blocks 240 for post-op have been amended, this may have an effect on the end time of the time segments corresponding to time blocks 210 for Patient 1.

In FIG. 2 only two hierarchical levels have been shown. It is possible to introduce a third level, a fourth level and even further hierarchical levels, each higher level having a composition link to the components of the just lower level.

As shown in FIG. 2, the time block 210 for Patient 1 has a relational link 390 with a time block 200 for a dressing room 1. Here again, an intersection between the time segments corresponding two time blocks 210 and 200 must be made, which may influence time segments according to both time block 210 and time block 200. Since time block 200 has no further sequential, composition or relational links, it is not necessary at this time to bring time segments according to time block 200 in accordance with time segments according to other time blocks, because it is in accordance with time block 210. However, since time segments according to time block 210 may have been changed and time block 210 has composition links to pre-op 220 and post-op 240, it may be necessary to amend the time segments according to time blocks 220 for pre-op or the time blocks 240 for post-op or both. Amendments to the time segments for time blocks 220 for pre-op may affect the time segments for time block 230 for exam, because of the sequential relation. The exam 230 must not start earlier than the earliest start time for pre-op plus the duration of pre-op and must not end later than the latest end time of pre-op plus the duration of the exam. As can be understood from the above description, taking into account the sequential, composition and relational links for making the time segments to correspond is a highly iterative and recursive task. In principle all time segments, whether for a patient, a dressing room, an apparatus, a radiologist, a nurse or whatever other resource, are equally treated. As soon as all the time segments in the system are made corresponding according to the time blocks, the system has found a “solution”. The “solution” which is most relevant here is made up of the time segments corresponding to time block 210 for Patient 1. Before offering the user to select a start time for the exam, it is useful to subtract the total duration of the components, i.e. pre-op, exam and post-op, from the end time of each segment corresponding to time block 210.

If the system has found a plurality of solutions, where each solution corresponds to another resource from one resource group or to another resource combination from two or more resource groups, then it is also advantageous to subtract from the end time of each of the time segments the duration of the exam, since that duration may be different for different resources within one resource group or for different resource combinations. These amended solutions can then be combined, by taking the union of the amended time segments over the different resources or combinations, and that union will correctly give the ranges of time intervals when the examination for the patient can take place.

The advantage of resource groups is that a resource group may be defined as a fixed group of resources, or by a technical constraint, such as the required capabilities of a CT device for performing the clinical examination. If a fixed resource group has 5 CT scanners, CT1, CT2, CT3, CT4 and CT5, then a sub-group of CT scanners can be defined as being those scanners that support a slice thickness of 1 cm. Such resource group may then comprise CT3, CT4 and CT5 only. A clinical examination that requires such operational constraint of a slice thickness of 1 cm, will thus receive available time segments that relate to CT3, CT4 and CT5 only. The list of available time segments will then preferably contain no single point where any of the time constraints or operational constraints is violated.

As an extension, the system may provide violation of at least one, some or all constraints, e.g. for emergency cases.

A specific room may be linked to a specific person by a constraint or by a preference. A preference may be superseded in case of an emergency for example.

Once the system has compiled all the available time segments, these time segments may be displayed for the user, such that he can select the most appropriate one. More specifically, the user may select a starting time for the appointment. The starting time is preferably within the interval of one of the available time segments. If the time segments give the complete available time, then the starting time is selected preferably in a time interval that starts at the start of the time segments and ends at the end of the time segment, minus the duration of the medical procedure. Preferably, the system displays for the user the individual available time segments as time periods in which the procedure or examination can start. Such time segment has a start time and an end time. During the period defined by the start time and the (end time+duration of the medical procedure), preferably all constraints are fulfilled. Even if the user selects the “end time” of the available time segment, the procedure can start on that “end time”, because the constraints are fulfilled until this end time plus the duration of the medical procedure.

If several time segments are available, it is preferred to display these time segments in a certain order. One of the options is to define preferences, and to display the most preferred time segments first. A preference is less strict than a constraint. A constraint is preferably not violated, whereas a preference gives a mere indication. Preferably, the constraints are evaluated first, resulting in the final list of available time segments. The preferences may be evaluated after evaluation of the constraints. Each preference may have an associated cost function. The global cost function may be the sum of the individual associated cost functions. The system may attempt to optimise, i.e. minimise or maximise, the global cost function, or use the evaluated global cost as a sorting criterion. An example of a preference may be a resource. If a patient is used to a specific physician or specific nurse, the user may prefer this physician or nurse, i.e. this resource. If a patient prefers to have the medical procedure on Wednesday rather than on Thursday, or a morning examination rather than an afternoon examination, this may be entered as a preference. If a hospital has several locations within one town or country, the user may enter the preferred location. Preferences may be entered by the user, or may be given by the hospital. A preference from a hospital may be that a specific physician preferably uses a specific examination room. If examination rooms are quite distant from each other, e.g. 500 metres, it may be useful to keep one person or a team of persons within one specific location, unless constraints require making an exception. Preferences can also relate to relative quality or capabilities of several devices. If a more recent device is not available, an older version of this device, having fewer capabilities, may also perform the examination. A quality or capability may be given as a constraint, if the quality or capability is really required, or it can be given as a preference, if the quality or capability is preferred only.

The resources may relate to a person, such as a physician, a nurse, an operator of a specific piece of equipment etc. A resource may also relate to the device or specific piece of equipment, a room where the medical procedure has to be performed or even a department. Typical resources or resource groups are radiologists, radiographers, rooms, echographic devices, CT or MR scanners.

By using the concept of the available time segments, the system makes sure that all constraints are fulfilled for these segments. The user can easily get more than one possible solution for the following reasons. Each available time segment gives at least one viable solution. If the available time segment stands for a duration longer than the required duration of the medical procedure, then the user can freely select several solutions within that time segment.

Moreover, by introducing the concept of preferences, the user gets a more suitable presentation of the available time segments. Prior art methods used a different searching strategy, and lost the continuity by making too early a discretisation step.

According to the current invention, the proposed solution gives the user a complete overview of all possible available time segments within a very large time scope or time window when an examination or group of examinations can take place, taking into account all operational constraints. An operational constraint may be that a certain medical examination must be done by a male person. In such case, the resource group for that resource is restricted to male persons.

Based on the proposed overview, the user can quickly react, without delay or any additional interaction with the software or communication with a server to see when an appointment or set of appointments of a particular type for a particular patient can take place at a particular time.

The overview of solutions is preferably presented as a list of segments of time. These segments may represent start time domains of valid appointments. This means that any point inside a segment is considered a valid start time for the appointment. The user can freely choose any start time as long as it belongs to any of the listed segments; the system may then ensure that a valid appointment can be made as such, preferably taking into account all operational constraints. This gives the user the ability to react quickly, for instance to special requests from a patient on the phone, and gives the user more control to choose an appointment that fits his needs best.

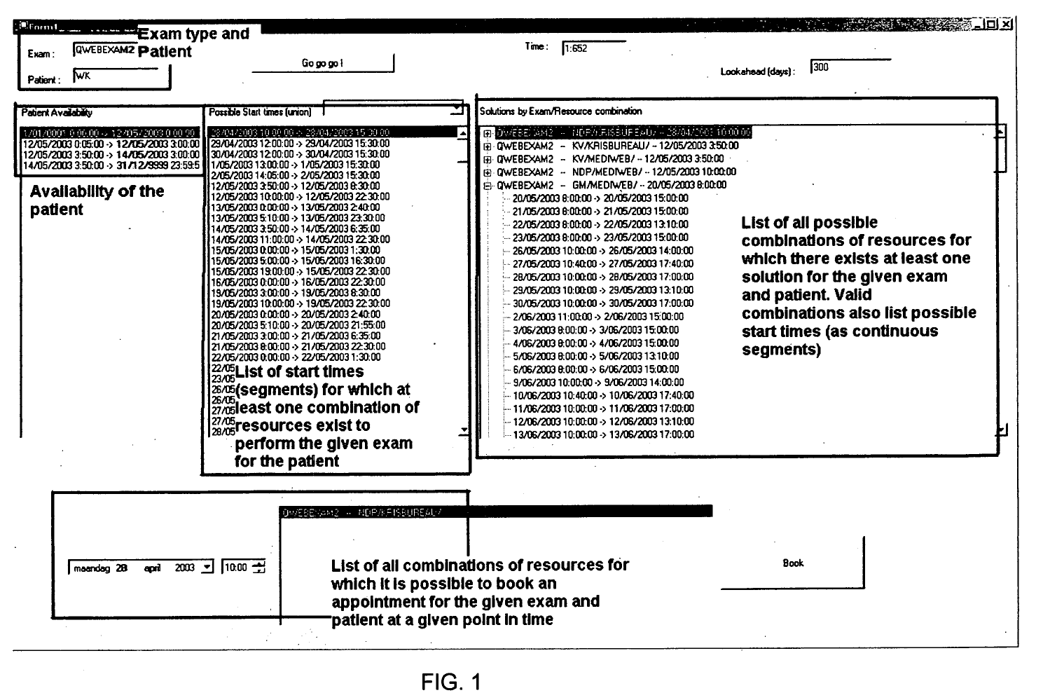

An example is shown according to FIG. 1.

If the system according to the current invention has to book an examination of type ‘QWEBEXAM2’ for a patient identified as ‘WK’, a system and method according to the invention preferably proceeds as follows.

QWEBEXAM2 is defined as an examination requiring two specialised resources. A resource may be one of the following items:

one person performing the examination; a location or room where the examination or procedure can take place; and, the medical equipment or medical device required for the procedure.

The examination constraints are preferably defined on particular qualities that resources must have to be valid candidates.

In order to provide a complete overview of all possible solutions for appointments in time, the system will take into account all availability parameters of all items involved, such as holidays of resources, working hours of resources, whether the resource is already scheduled at times, general holidays, closing hours, availability of the patient, and so on.

As shown in FIG. 1, upon entering the examination ‘QWEBEXAM2’ and the patient code ‘WK’ and clicking a button, the user gets the following information on the screen.

On the upper right hand of the window in FIG. 1, the box “Lookahead (days)” specifies the time window in which the available time segments will be searched.

The column “Availability of the patient” gives the time window for the patient or his “available personal time segment”. That time window comprises various available personal time segments, four in this example. Each time segment gives a start time and an end time. The start time is identified by a date and time, with a precision up to one second. That time thus specifies the hour, minute and second. The end time is specified by date and time, just as the start time.

The second column “List of start times (segments) for which at least one combination of resources exist to perform a given exam for the patient” lists the available time segments. Each line gives an available time segment within the time window specified in the “Lookahead” box. The available time segments are sorted in chronological order. In this example, the available time segments are disjunctive, which means that there is no time overlap between any two different time segments.

The third column of FIG. 1 lists all possible combinations of resources for which there exists at least one solution for the given examination and patient. These valid combinations also list the possible start times, as continuous time segments. As can be noticed, the list in this third column gives the time segments for which both the patient is available and the constraints for column 2 are fulfilled. The user may then select one of the segments displayed in column 3, in this case the top line. This selection is then displayed at the left lower portion of the window, saying Monday 28 Apr. 2003 10:00. This segment will then be copied to the box below and be entered in the box that lists all combinations of resources for which it is possible to book an appointment for the given exam and patient at a given point in time. At this time, the user may click the “Book” button, such that the appointment is scheduled.

As such, the various lists in FIG. 1 give the user the ability to quickly identify validity for an appointment and to select the most appropriate time. For instance, the middle column lists all time segments when the appointment can start taking into account all constraints of the patient, its availability and possible involved resources. The system provides that at least one combination of resources is available for any point in time listed in the middle column.

The system will propose a time and resource combination for the appointment based on quality criteria that can be configured by the medical institution, however, the user can easily modify the proposed time and resource combination. Consequently, the user may book the appointment by clicking a button.

Having described in detail preferred embodiments of the current invention, it will now be apparent to those skilled in the art that numerous modifications can be made therein without departing from the scope of the invention as defined in the appending claims.

1. A method for scheduling an appointment comprising the following steps:
specifying a time window for said appointment;
searching for an available time segment within said time window;
selecting a most appropriate starting time for said appointment within said available time segment.

2. The method according to claim 1 comprising the steps of:
specifying said time window by entering at least a start time in a first computer system and storing this start time in a storing device;
specifying at least one resource, required for said appointment;
identifying at least one available resource time segment, associated with said resource;
storing said available resource time segment in a second computer system, coupled to said first computer system; and,
identifying said available time segment as a time segment comprised in said available resource time segment.

3. The method according to claim 1, comprising the steps of:
specifying at least one first resource group, said first resource group comprising a plurality of first resources, one of which being required for said appointment;
for each of said first resources, identifying at least one available resource time segment, associated with said first resource;
for each of said first resources, identifying said available time segment, comprised in said available resource time segment;
after selection of the most appropriate starting time, selecting the first resource for which the starting time is comprised in the associated available resource time segment.

4. The method according to claim 3, comprising the steps of:
specifying a second resource group, said second resource group comprising a plurality of second resources, one of which being required for said appointment;
for each of said second resources, identifying at least one available resource time segment, associated with said second resource;
for at least one combination of one of said first resources and one of said second resources, identifying said available time segment as being comprised in said available resource time segment associated with said first resource and in said available resource time segment associated with said second resource;
after selection of the most appropriate starting time, selecting the combination of said first resource and second resource for which the starting time is comprised in the associated available resource time segments for the first and second resource.

5. The method according to claim 1, further comprising the steps of:
defining at least one preference for said appointment;
finding a plurality of available time segments; and,
arranging said time segments according to said preference.

6. The method according to claim 5, wherein said arranging is selected from the group consisting of:
sorting; and,
computing a cost factor.

7. A system for scheduling an appointment comprising:
means for specifying a time window for said appointment;
means for searching for an available time segment within said time window;
means for selecting a most appropriate starting time for said appointment within said available time segment.

8. A method for scheduling an appointment using a first resource and a second resource comprising:
initialising first data for said first resource having first available resource time segments;
initialising second data for said second resource having second available resource time segments;
defining a relational link between said first data and said second data for identifying simultaneous action of said first resource with said second resource;
amending said first data and said second data until each time segment represented by said first data equals to just one time segment represented by said second data;
selecting said available time segments based upon one of said amended first and second data.

9. The method according to claim 8, further comprising:
initialising third data for a third resource having third available resource time segments;
defining a sequence link between said first data and said third data for identifying a sequential action of said first resource by said third resource;
amending said first data and said third data until each time segment represented by said first data is preceding at least a portion of one time segment represented by said second data.

10. The method according to claim 8, further comprising:
initialising fourth data for a fourth resource having fourth available resource time segments;
defining a composition link for linking said first resource and said fourth resource in one entity;
amending said first data and said fourth data until both fit in said entity.
